(19) United States
(12) Patent Application Publication
(10) Pub. No.: US 2018/0043059 A1
(43) Pub. Date: Feb. 15, 2018

(54) 3D Ti-6Al-4V Structures with Hydrogel Matrix

(71) Applicants: Victor Rodriguez Correa, El Paso, TX (US); Lawrence E. Murr, El Paso, TX (US); Kristine Garza, El Paso, TX (US)

(72) Inventors: Victor Rodriguez Correa, El Paso, TX (US); Lawrence E. Murr, El Paso, TX (US); Kristine Garza, El Paso, TX (US)

(73) Assignee: THE BOARD OF REGENTS OF THE UNIVERSITY OF TEXAS SYSTEM, Austin, TX (US)

(21) Appl. No.: 15/672,556

(22) Filed: Aug. 9, 2017

Related U.S. Application Data
(60) Provisional application No. 62/372,415, filed on Aug. 9, 2016.

(51) Int. Cl. A61L 27/52; A61L 27/06; A61L 27/14; A61K 31/16; A61L 27/22; A61L 27/54; A61L 27/56

(52) U.S. Cl. CPC A61L 27/52; A61L 27/54; A61L 27/06; A61L 27/56; A61K 31/16; A61L 27/227; A61L 27/14; A61L 2430/02; A61L 2300/414; A61L 2300/232

(57) ABSTRACT

Embodiments of the invention are directed to a vascular structure forming implant produced by additive manufactured Ti-6Al-4V foams a living implant.

### CROSS REFERENCE TO RELATED APPLICATIONS

This Application claims priority to U.S. Provisional Applications 62/372,415 filed Aug. 9, 2016, which is incorporated herein by reference in its entirety.

### BACKGROUND OF THE INVENTION

### A. Field of the Invention

The invention generally concerns a hydrogel and methods of using the same. In particular the hydrogel is provided in a Ti-6Al-4V foam or scaffold.

### B. Description of Related Art

3D printing or Additive Manufacturing (AM) has revolutionized the way materials scientists and engineers synthesize a broad spectrum of materials. This technology allows bioengineers to enhance metals, composites, polymer plastics, and biomedical devices. Tissue engineering is an emerging field in which materials that display biomimetic properties are employed for medical applications. Despite the considerable amount of research advances being made (Surmenev et al., Acta Biomaterialia, 2014, 10:557-79; Bosch et al., Journal of Craniofacial Surgery, 1998, 9:310-6; Meinel et al., Bone, 2005, 37:688-98; Gugala and Gogolewski, Injury, 2002, 33:71-6; Schütz and Südkamp, Journal of orthopaedic science, 2003, 8:252-8; Kumar et al. Materials Science & Engineering R: Reports, 2016, 103:1-39), the main challenge in tissue engineering is to create a fully functional living system from a non-living scaffold. There exists an ever present need to develop materials that are not just bio-compatible, but that can more closely mimic a complete biological system.

Living tissue is comprised of complex interactions between different cell types, all of which perform different tasks, depending on the cell type and the type of tissue. Due to highly regulated cell biology processes, complexity of molecular interactions and cellular differentiation hierarchy, engineering tissue remains a challenging endeavor. Bone is composed mainly of mineralized calcium crystals (hydroxyapatite), with a chemical formula of Ca₅(PO4)₃(OH), and collagen. The mixture of these structures provides mechanical support and a degree of elasticity (Kumar et al., International Materials Reviews, 2016, 61:20-45). Despite the success of implanted orthopedics, mainly hip-replacement implants, there has been evidence of infection, aseptic loosening, pain without loosening or other reasons of failure (Diefenbeck et al., Biomaterials, 2011, 32:8041-7). When a bone replacement implant is inserted, a surgeon needs to cut an extensive amount of bone, effectively creating a wound. Cells at the interface between the bone and the implant cannot grow into the solid bio-compatible metal alloy. Bone replacement implants remain as solid structures, providing a physical limitation for cells to grow.

### SUMMARY

Many strategies have been employed in order to enhance the bio-compatibility of orthopedic implants (Surmenev et al., Acta Biomaterialia, 2014, 10:557-79; Bosch et al., Journal of Craniofacial Surgery, 1998, 9:310-6; Meinel et al., Bone, 2005, 37:688-98; Gugala and Gogolewski, Injury, 2002, 33:71-6; Schütz and Südkamp, Journal of orthopaedic science, 2003, 8:252-8; Kumar et al. Materials Science & Engineering R: Reports, 2016, 103:1-39), but a more diverse set of materials needs to be examined in order to closely mimic a complete living system. To this end, 3D printed structures, e.g., foams or scaffolds, are an effective alternative, given their degree of porosity, in particular Ti-6Al-4V printed foams or scaffolds. Advantages to this approach include varying pore size gradient, varying porosity, and a high degree of resolution control on the implant synthesis. An extracellular matrix-like gel in combination with 3D printed foams or scaffolds was evaluated for the development of a bone replacement implant. In this research, a Ti-6Al-4V structure was designed to promote cell migration of vascular endothelial cells, and differentiation and proliferation of pre-osteoblast cells. Given that there is a degree of porosity in these structures, a matrix can be applied to the structure, allowing for microcapillary formation in a 3D suspension. Since hydrogels are highly hydrated polymers, certain molecules and growth factors can be mixed into it, providing cells with the necessary supplements required for proliferation and growth.

Certain embodiments are directed to a living implant. A living implant is one that creates a living replacement of bone tissue. In certain aspects a Ti-6Al-4V structure can be used in combination with a hydrogel material containing a stress inducer. Portions of the hydrogel will be catalyzed, metabolized, or degraded by surrounding tissue and eventually replaced by said tissue. An argument can be made that the ingrowth of bone presents an answer to stress shielding effects of modern day implants. A stress inducer can enhance the production of hydroxyapatite and bone density should not decrease as a result.

Hydrogels have been widely employed in cell culture environments, with varied applications (Kumar et al., Journal of biomaterials applications, 2016, 0885328216658376; Kumar et al. Materials Science and Engineering C, 2012, 32:464-9). Many materials have been examined and tested for applications in the biomedical field, but one of the most promising materials has been gelatin based hydrogels (Kumar et al. Materials Science and Engineering C, 2012, 32:464-9; Yuksel et al., International Journal of Pharmaceutics, 2000, 209:57-67; Kumar et al., Journal of Biomedical Materials Research Part A, 2013, 101:2925-38). Developing a 3D matrix to grow cells in is advantageous in many ways, given that it has been previously shown (Kumar et al., Journal of biomaterials applications, 2016, 30:1505-16) that a 2D environment does not fully mimic physiological conditions. This is because the 3D matrix will allow cells to behave as they would in a normal physiological environment morphologically and also enhancing cell-cell communication (Rowley et al., Biomaterials, 1999, 20:45-53).

Certain embodiments are directed to an implant or a bone replacement implant comprising (a) a three dimensional support; and (b) a hydrogel matrix comprising a hypoxia inducer and glucose, wherein the implant is capable of promoting vascularization and osteogenesis. In certain aspects the three dimensional support is a Ti-6Al-4V structure or similar alloy. The structure can have a porosity of 50 to 70%, in particular 60%. The structure can have an average pore size of 200, 300 to 400, 500 μm, in particular about 350 μm. In certain aspects the structure has a thickness of 0.25 to 5 cm. The density of the structure can be between 0.5 to 3 g/cm² or 1 to 2 g/cm² or in particular about 1.5 g/cm². In certain aspects the hypoxia inducer is deferoxamine mesylate (DFM). DFM can be present in the hydrogel at a concentration of about 0.5, 1, 2 to 5, 10, 15 μM, including all values and ranges there between. In certain aspects DFM concentration can be as high as 2 to 10 mM. The hydrogel can comprise natural, synthetic, or natural and synthetic polymers. In certain aspects the hydrogel can comprises proteins of the extracellular matrix, particularly collagen. Natural polymers can include one or more of polyhyaluronic acid, alginate, polypeptides, collagen, elastin, polylactic acid, polyglycolic acid, or chitin. Synthetic polymers can include one or more of methacrylated gelatin, polyethylene oxide, polyethylene glycol, polyvinyl alcohol, polyacrylic acid, polyacrylamide, poly(N-vinyl-2-pyrrolidone), polyurethane, or polyacrylonitrile. In certain aspects the hydrogel further comprises one or more growth factors or antibiotics.

A metal or alloy foam is a cellular structure consisting of a solid metal (frequently aluminium) with gas-filled pores comprising a large portion of the volume. The pores can be sealed (closed-cell foam) or interconnected (open-cell foam). The defining characteristic of metal foams is a high porosity: typically only 5-25% of the volume is the base metal, making these ultralight materials.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. EBM fabricated Ti-6Al-4V structure.

FIG. 2. Comprehensive MTS viability Assay of MC3T3-E1 pre-osteoblast cells exposed to both CoCl₂ and DFM.

FIG. 3. Viability test measuring response of HUVECs and MC3T3-E1 cells to DFM, D(+) glucose and DFM+D(+) glucose.

FIG. 4. Trypan blue exclusion dye cellular proliferation analysis for MC3T3-E1 cells exposed to DFM and CoCl₂ for 24 hours.

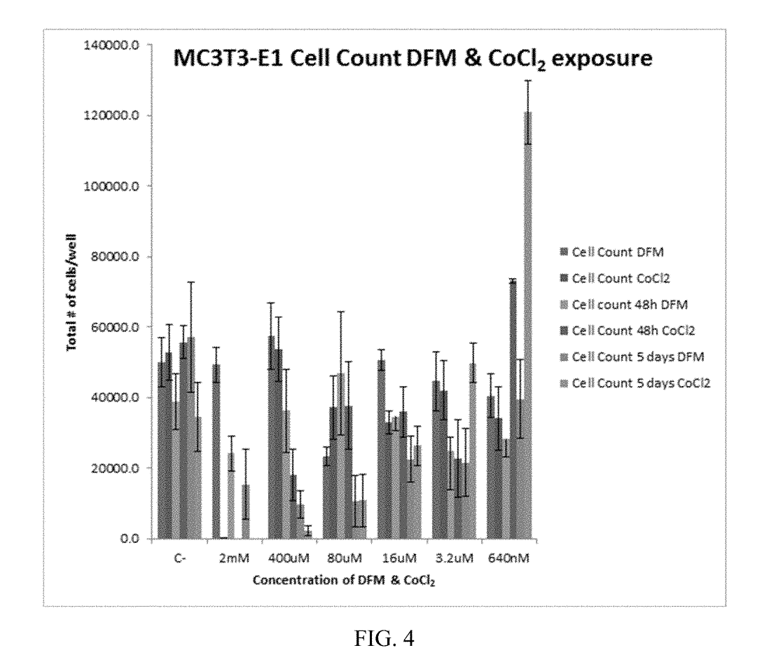

FIG. 5. Alizarin Red S staining assay performed on MC3T3-E1 cells exposed to various concentrations of DFM.

FIG. 6. ARS analysis performed on MC3T3-E1 cells grown in foams for 7, 14 and 21 days. Negative controls represent cells without DFM exposure. Experimental setting represents cell exposed to 3.2 μM DFM.

FIG. 7. Vascular network formation on foams and Matrigel mix under DFM treatment. HUVECs (left and center) & MC3T3-E1 (top right).

### DESCRIPTION

Foams provide an ideal substrate to substitute bone due to their random distribution and interconnection, which is largely similar to that of real bone. The foam is a composite of pores, struts and nodes. The struts connect nodes of the material in three dimensions to create a collection of pores, or cages (or also referred to as a scaffold). The pores may be open or closed, as in open-cell and closed cell foams. Ti-6Al-4V has been a popular alloy used in the biomedical industry and research and has been extensively characterized (Murr et al., Int J Biomater. 2012, 2012:245727; Bertassoni et al., Biofabrication. 2014, 6(2): 024105; Rivrona et al., PNAS, 2012, 109(18):6886-91; Klein-Nulend et al., European Cells and Materials 2012, (24) 278-91). The limitation of free iron availability through exposure of DFM seems to be a driving factor to enhance the synthesis of hydroxyapatite by cells. It has been previously demonstrated that pre-osteoblasts proliferate, differentiate and are able to synthesize hydroxyapatite when grown on foam and mesh structures of this alloy. However, described herein for the first time is an implant that supports formation of a vascular network in the context of a foam alloy.

The process of vascular structure initiation has a key step that involves proteolytic degradation of the ECM so that endothelial cells can migrate to form the microcapillarities (Victor, et al., Cardiovascular Research 2008, 78:203-12). DFM has proven to be a suitable candidate molecule to promote vascularization of endothelial cells. Immediate biomedical applications of this iron chelating agent are viable, seeing as it is already been FDA approved for the treatment of iron poisoning. Described herein is the concept of a living implant as it pertains to various cellular molecular mechanisms, mainly involved in wound healing and the regeneration of tissue. Tissue that has undergone extensive damage needs to endure harsh environments that stimulate apoptosis rather than cell survival. A tissue-solid metal interface is not sufficient to promote a wound healing process, the ingrowth of bone, and eventually the formation of a vascular network. Implanted solid metal bars present a physical limitation to the availability of nutrients and, most notably, oxygen. The wounded tissue then suffers from hypoxia, triggering an irreversible response that eventually leads to cellular death. In a heavily wounded tissue metabolic demands differ vastly from that of normal tissue. To create a fully living implant that mimics real tissue, this issue needs to be addressed and thoroughly understood. Therefore, the addition of molecules that can compensate for the metabolic high demands is required. It is to this end that D(+) glucose can be added to cells undergoing a hypoxia mimetic response.

The materials chosen and tested herein prove to be a combination that is suitable to develop a fully living bone replacement implant. Ti-6Al-4V foams provide structural support, while an ECM-like hydrogel simulates an aqueous microenvironment that drives wound healing, bone ingrowth and vascularization. Despite the attractive properties of Matrigel, this product is not intended for anything other than research purposes. However, its main constituents may be further utilized with the focus of creating a hydrogel capable of driving the before mentioned processes. Collagen and gelatin hydrogels can be tailored to maintain their solid stability under physiological conditions (Bertassoni et al., Lab Chip, 2014, 14:2202; Rassu et al., Carbohydrate Polymers 2016, 136:1338-47).

Embodiments of the invention include materials comprising a base support coupled to a hydrogel that includes reagents for supporting vascularization and enhancing bone repair, while maintaining mechanical and structural similarities with real bone. Certain aspects are directed to a mixture of additive manufactured Ti-6Al-4V foam in combination with a collagen based hydrogel matrix containing DFM; a hypoxia mimetic compound, that can form vasculature under physiological conditions, while maintaining osteoblast cell differentiation and proliferation. This approach induces a hypoxia mimetic stress that will trigger cellular survival signals that ultimately enhances wound healing processes in bone.

### A. Structured Foam Support

A structured foam can be rapidly built from a base powder material. For example, the foam structure can be manufactured using three-dimensional (3D) printing. A direct metal laser sintering process can be used to 3D print (i.e. build) the foam structure. The foam structure can be made from a base material, such as a Ti-6Al-4V alloy. In other embodiments the foam structure can comprise other suitable alloys or combination of alloys (e.g., 316 stainless steel, commercially pure titanium (TiCP) and aluminum alloy (AlSi₁₀Mg), austenitic steels, ferrous steels, aluminum alloys, titanium alloys, pure aluminum and pure titanium and the like).

The foam structure can be three-dimensionally printed with a direct metal laser sintering process (or any other suitable process, such as electron beam melting). The foam structure can be three-dimensionally printed with at least a Ti-6Al-4V alloy (or any other suitable alloy or combination of alloys). The foam structures can be produced using electron beam melting or any other additive manufacturing process.

### B. Extracellular Matrix-Like Hydrogels for Bone Repair

The ingrowth of bone into the implant is essential in order to achieve what is conceptually a living implant. Although the main goal of this research is to stimulate the formation of vascular structures within the porous metal implant, the nature of wound healing must also be addressed. This includes, but is not limited to the mineralization of calcium by osteoblasts, the inhibition of bone resorption by osteoclasts, and avoiding debris release by the implant itself. Bone has elastic properties, and its elasticity can be attributed to the collagen in which hydroxyapatite grows. The molecular arrangement of collagen is regulated by fibroblasts and endothelial cells in tissue (Xie et al., Calcif Tissue Int 2016, 98:275-83), and this arrangement directs the synthesis and growth of hydroxyapatite. Bone is capable of self-repair, but this natural process is limited to the extents to which it can generate new tissue. This is the case for the large portion of bone that has been surgically removed. However, with the assistance of engineered biomaterials, bone tissue repair can be directed by stimulating the appropriate wound healing response. The microenvironment of bone has been widely reported to be hypoxic (Guo et al., PLoS ONE 10(11): e0139395). A hypoxia mimetic environment has been reported to enhance bone repair, along with restoring endothelium integrity (Nune et al., J Biomed Mater Res Part A 2014, 00A:000-000). This was determined by injecting DFM on mandibular fractures that had been exposed to radiotherapy. DFM improved healing and augmented vascularity. Iron chelation by DFM administration has also shown that bone resorption is inhibited by limiting osteoclastic differentiation (Gaytan et al., Metallurgical and Materials Transactions A. 2010, 41(12):3216-27; Murr et al., Journal of the Mechanical Behavior of Biomedical Materials. 2009, 2(1):20-32). It is to this end that a collagen based hydrogel material is ideal to, not only serve as a mimetic of an ECM, but to also serve as an aqueous solution in which to deliver hypoxia mimetic inducing compounds such as DFM.

The implementation of a hydrogel matrix also eliminates the issue of seeding efficiency of cells into the foam structure. When cells are added to the structure, they will be in a liquid suspension that will eventually become a solid hydrogel matrix. Because the proposed model will have a solid matrix, cells will not fall through the porous metal foam at the moment of seeding. Instead, they will remain suspended in the ECM-like gel.

Certain embodiments are directed to design a bone replacement implant capable of forming vascular structures in a hydrogel matrix, while allowing for osteoblast proliferation and cell differentiation. Osteoblasts can also successfully synthesize hydroxyapatite and retain their adhesion to the Ti-6Al-4V foam (Murr et al., J Mech Behav Biomed Mater. 2011, 4(7):1396-1411). The hydrogel matrix should contain all of the necessary supplements to favor angiogenesis and vascular structure maturation.

A hydrogel is a three dimensional network of polymer chains with water filling the void space between the macromolecules. In certain aspects the hydrogel includes a water soluble polymer that is crosslinked to prevent its dissolution in water. The water content of the hydrogel may range from 20-80%. In certain aspects the hydrogel may include natural or synthetic polymers. Examples of natural polymers include polyhyaluronic acid, alginate, polypeptide, collagen, elastin, polylactic acid, polyglycolic acid, chitin, and/or other suitable natural polymers and combinations thereof. Examples of synthetic polymers include polyethylene oxide, polyethylene glycol, polyvinyl alcohol, polyacrylic acid, polyacrylamide, poly(N-vinyl-2-pyrrolidone), polyurethane, polyacrylonitrile, and/or other suitable synthetic polymers and combinations thereof. For example, the hydrogel may include a crosslinked blend of polyvinyl alcohol (PVA) and poly(N-vinyl-2-pyrrolidone) (PVP). The hydrogel may also include beneficial additives that are released at the surgical site. For example, the hydrogel may include analgesics, antibiotics, growth factors, and/or other suitable additives.

### C. Angiogenesis

The process of the development of new vasculature (angiogenesis) is one component of the wound healing process (Ramasamy et al., Nature 2014, 507:376-80). Vascular structures serve as transport pathways for oxygen, nutrients and signaling molecules throughout the organismal systems. Because of the significance of this process, the capacity of implant to induce vascularization is essential to develop an ideal substitute of the original biological matter. It has been recently studied that cell-cell differentiation in developing organs is key for the development of angiogenesis (Yancopoulos et al., Nature 2000, 407; Novosel et al, Advanced Drug Delivery Reviews 2011, 63:300-11). These interactions are mediated by Endothelial Cells (EC). It is these cells that form the first liner that becomes the basic template for the formation of veins and arteries. The main role of an endothelium is to serve as a transport pathway for oxygen. Therefore, ECs are equipped with oxygen sensor molecules such as Prolyl Hydroxylase Domain Enzymes (PHDs), and Hypoxia Inducible Factors-1α (Hif-1α). Despite the biological importance of vascular structure formation, achieving this remains a challenge in tissue engineering (Carmeliet and Jain, Nature 2011, 473).

Angiogenesis can be initiated by certain growth factors, the most widely acknowledged signaling pathway being triggered by Vascular Endothelial Growth Factor (VEGF). Research has demonstrated that certain proteins, regulate the levels of VEGF secretion and play key roles in angiogenesis (Li et al., Sci Rep. 2015, 5:12410), the most important of these being the Hif-1α. Hif-1α acts as a transcription factor, translocating to the cell's nucleus under depravation of oxygen. This transcription factor increases the number of type HECs and osteoprogenitors through the process of hypoxia (Yue et al., Biomaterials 2015, 73:254e271).

Hypoxia is defined as the deficiency of oxygen in tissues. When oxygen is depleted in tissue, a highly regulated process concerning cell survival becomes activated. Hif-1α is highly down-regulated by PHD-2 which target Hif-1α for degradation. During hypoxia, there is lack of oxygen in cells, which inactivates the prolyl hydroxylase domain proteins PHD1-3, which are oxygen-sensing (Jaakkola et al., Science. 2001, 5516:468-72). When this occurs, Hif-1α and Hif-2α proteins are no longer targeted for protein degradation and transcriptional responses are then activated to increase oxygen supply by angiogenesis through upregulation of VEGF (Kusumbe et al., Nature 2014, 507:323-28; Aro ey al., J Biol Chem. 2012 287(44): 37134-44). In general, Hif-1α promotes vessel sprouting, whereas Hif-2α mediates vascular maintenance (Kusumbe et al., Nature 2014, 507:323-28; Aro ey al., J Biol Chem. 2012 287(44): 37134-44). Hif-1α abrogation by siRNAs in HUVECs disrupts the formation of microcapillaries, but not Hif-2α (Veschini et al., Blood 2007 109(6)). This is because Hif-2α does not stimulate the production of VEGF (Veschini et al., Blood 2007 109(6)).

ECs migrate to reorganize themselves under hypoxia (Victor, et al., Cardiovascular Research 2008, 78:203-12). The secretion of VEGF stimulates this reorganization. When VEGF is secreted, ECs also secrete metalloproteinases, whose role is to rearrange the ECM (Mori et al., The EMBO Journal 2002, 21(15):3949-59). After the ECM rearrangement, they begin to express CD44, allowing for an increased cell adhesion (Kim et al., Immunology, 129:516-24) that enables the endothelium to maintain is microcapillar structure. Despite the high level of cellular organization to form microcapillaries, microvessel maturity is an issue as well. When microcapillaries form, endothelial cells may become quiescent (increased cellular half-life). However, the microsvascular structure may not always be retained, unless the endothelium recruits a pericyte liner. Pericytes are recruited by the endothelium when endothelial cell quiescence is achieved, which is determined by the secretion of Angiopoetin 1 & 2 (ANG-1 & ANG-2). The secretion of ANG-1 signals endothelium quiescence, whereas ANG-2 is secreted by Endothelial Tip Cells (ETCs) (Li et al., Sci Rep. 2015, 5:12410). An ETC is a single endothelial cell randomly selected to commence the progression of a sprouting microvasculature. This process promotes vascular branching. In a physiological environment, ECs are held together by the Extracellular Matrix (ECM). This is a matrix represents a physical barrier that the endothelium can manipulate (Victor, et al., Cardiovascular Research 2008, 78:203-12).

It has been previously reported that hypoxia induced by exposing cells in vitro and in vivo to CoCl₂ causes severe inflammatory response, resulting in the recruiting of macrophages (Zhang et al., PLoS ONE 2013, 8(12):e84548). This has been observed in failed implanted structures that consist mainly of Cobalt-Molybdenum-Nickel alloys (Unger et al., Advanced Drug Delivery Reviews 2015). In this particular research, particulate debris from the implanted alloy was analyzed against macrophages, resulting in hypoxia. The authors analyzed the effects of Cobalt ions on cells, but did not evaluate hypoxia mimetic cellular response with anything other than Cobalt based materials. Despite these results, there have been a myriad of results demonstrating that hypoxic stress does not mediate cell death, instead, it promotes cell survival (Liu et al., Toxicol Appl Pharmacol. 2012, 264(2):212-21). It has been widely studied that Cobalt ions can stimulate the production of Reactive Oxygen Species (ROS), thus leading to mitochondrial insult, resulting in apoptosis (Unger et al., Advanced Drug Delivery Reviews 2015; Snyder and Chandel, Antioxidants & Redox Signaling 2009, 11(11)). This leads to a controversial issue: does a hypoxia mimetic environment necessarily cause an undesirable response in wound healing?

### D. Hypoxia in Wound Healing

Cells have evolved to respond to varied environments. Lack of free oxygen is one of them. Because oxygen is required for many cellular metabolic processes, such as the production of Adenosine Triphosphate (ATP), fatty acid synthesis and oxidative phosphorylation, cells are prepared to activate transcription factors that promote cell survival (Warnecke et al., The FASEB Journal express 2003, 10.1096402-1062fje). Under a hypoxic response, the Hif-1α intracellular levels increase, as it is no longer targeted for degradation by PHD enzymes (Alvarez-Tej ado et al., The Journal of Biological Chemistry. 2001, 276(25):22368-74). Hif-1α can then dimerize with Hif-1β in the cell nucleus and initiate the transcription process that results in the expression of the VEGF gene. VEGF has been reported to promote an angiogenic response, and increase the activation of the Phosphatidyl Inositol-3-Kinase (PI3K)-Akt signaling pathway (Chen et al., Tissue Engineering: Part A 2013, 19(19 and 20)). It has been broadly researched and acknowledged that this particular signaling pathway is actively involved in the progression of tumor invasiveness and metastasis in a variety of cancer models (Stegen et al., Cell Metabolism 2016, 23:265-79). Hypoxia has been reported to increase the viability of cells and progression of survival signaling pathways (Chen et al., Tissue Engineering: Part A 2013, 19(19 and 20); Liu et al., Journal of Inorganic Biochemistry 2016 online). However, on a normal cell line, inhibiting the degradation of Hif-1α inhibits apoptosis, does not produce ROS (as Cobalt does), but results in promoting cellular differentiation and migration. Moreover, because the PI3K-Akt pathway becomes activated while a cell is experiencing a hypoxic response, therefore, diligent care must be taken in order to, not only select an appropriate hypoxic inducer, but to employ it at the correct concentrations. Despite the molecular signaling similarities between hypoxia stressed cells and cancer, the metabolic profiles of each are different (Zhanga et al., Toxicology and Applied Pharmacology 2016, 301:50-60). This suggests that, though the PI3K/AKT pathway is expressed, no adverse effects such as the immortalization of cells should be observed. The viability, proliferation, and population doublings of the cells exposed to various hypoxia inducing molecules must be addressed, and must not be limited to endothelial cells.

Deferoxamine Mesylate (DFM), also referred to as Deferoxamine (DFO) is an iron chelating agent; meaning that it binds to free iron ions in solution. This particular molecule is employed to regulate iron homeostasis in cells by chelating excess iron in solution (Huang et al., Cell Signal. 2014, 26(12):2702-09; Chachami et al., Am. J. Respir. Cell Mol. Biol. 2004, 31:544-51). DFM is a well know inhibitor of PHD enzymes and has also been shown to increase bone density in osteoporosis mouse models (McDonough et al., PNAS 2006, 104(26)). Despite there being other chemicals that may induce hypoxia in cells, i.e. CoCl₂ (Selvaraju et al., Antioxidants & Redox Signaling 2014, 20(16)), DFM has little known adverse effects.

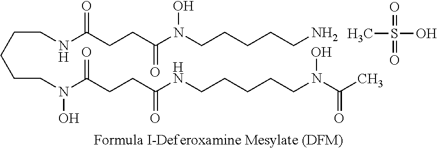

Because DFM binds to iron co-factors, the catalytic ability of PHD enzymes becomes hindered, leading to the stabilization of Hif-1α. DFM has been approved by the Food and Drug Administration (FDA) and is available for clinical use in the US. Currently, it is being used as an iron chelating agent to treat iron overdose from blood transfusions. As previously mentioned CoCl₂ triggers a hypoxic response and stabilized Hif-1α because it competes with iron for enzymatic active sites.

In recent years a wide number research papers have been published with promising applications for hypoxia in wound healing (Zhanga et al., Toxicology and Applied Pharmacology 2016, 301:50-60; Karuppagounder et al., Science Translational Medicine 2016, 8(328)). These approaches include but are not limited to diabetic wound healing in fibroblasts (Ehrbar et al., Biophysical Journal 2011, 100:284-93), several mitochondrial related metabolic diseases such as Leigh Syndrome (Asosingh et al., Haematologica 2005, 90:810-17), and more recently to treat brain hemorrhage (Donneys et al., Bone. 2013, 52(1):318-25). The biomedical applications of hypoxia can be tailored to combat a variety of wound healing situations. It has also been recently reported that inhibiting PHD2 enzymes and stabilizing Hif-1α increases the survival rate of newly implanted cells in bone (Zhanga et al., Toxicology and Applied Pharmacology 2016, 301:50-60). It has been reported that the levels of ROS species in endothelial cells decreases, enabling cells to undergo redox homeostasis and glycogen storage. This further suggests that a hypoxia mimetic, but not hypoxia as a lack of oxygen maintains the integrity of cellular metabolism by stabilizing Glutathione S Transferase (GST). Because of the ever increasing evidence that hypoxia can support regenerative medicine, in this research, a hypoxia mimetic will be applied to promote vascularization, pre-osteoblast differentiation and wound healing for newly implanted bone replacement implants.

### E. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

1. Materials & Methods

Material Fabrication, Preparation and Characterization.

An interconnected foam structure of Ti-6Al-4V was micro-fabricated by Electron Beam Melting (EBM) techniques. The spherical powder size that was used was in the range of 30 μm. An ARCAM-EBM system was used for the fabrication of foam structures. The density was determined by using the Archimedes Principle for sintered powder metallurgy products ASTM B962-13.

The foam structures fabricated via the EBM system were qualitatively characterized by scanning electron microscopy (SEM) and quantitatively through a densitometry approach. The pore size was estimated from the SEM micrographs, whereas, porosity and modulus were calculated from densitometric measurements using the following equations:

$$\% Porosity = [ 1 - \frac{ρ}{{ρ}_{0}} ] \times 100$$

$$Gibson \text{-} Ashby equation$$

$$\frac{E}{{E}_{0}} - {( \frac{ρ}{{ρ}_{0}} )}^{n}$$

where E and Eₒ are the stiffness for an open-cellular structure and solid (fully dense) material having a density of ρ and ρ₀, respectively. For Ti-6Al-4V alloy, Eₒ=110 GPa, ρ₀=4.43 g/cc, n varies from ˜1.8-2.2, and can be approximated to be 2.

Implementation of a Hydrogel Matrix on the Ti6-4Al-V Foam.

In order to produce a fully functional orthopedic implant, an aqueous matrix needs to be added to the printed foam structures. Cells may grow freely in this structure so they may behave as they would in normal tissue. The cell biology of angiogenesis is a tightly regulated and complex process, orchestrated only when necessary. A myriad of cell types, such as endothelial cells, pericytes, osteoblasts, etc. have roles to fulfill in the process off wound healing in bone, all of which need to interact with each other, differentiate and migrate in three dimensions for complete wound healing. The process of wound healing includes the re-establishing of osteoblast mediated synthesis of calcium oxalate, the ability of endothelial cells to form a microvasculature and ensuring the maturity and stability of said vasculature.

Given that there is such a high level of cellular organization in the process of forming new vasculature, the aqueous matrix employed in this research needs to be as similar to an Extra Cellular Matrix (ECM) as possible. To this end, Corning® Matrigel® Matrix was evaluated as a potential matrix, given that its main constituents are ECM proteins such as laminin, collagen IV, heparin sulfate proteoglycans, entactin/nidogen, and a number of growth factors.

Cell Culture.

The MC3T3-E1 Subclone 4 (mouse pre-osteoblasts (ATCC® Manassas, Va., USA CRL-2593™)) cell line was used as a pre-osteoblast model. MC3T3-E1 Subclone 4 were grown in α-MEM (Alpha Minimum Essential Medium) (Sigma-Aldrich) cell medium supplemented with 10% FBS (Fetal Bovine Serum) (ATCC 30-2020). Human Umbilical Vein Endothelial Cells (HUVECs), (ATCC® Manassas, Va., USA CRL-1730™) are grown in 1:1 F-12K/DMEM media supplemented with 10% FBS, 0.1 mg/ml heparin; 0.03-0.05 mg/ml endothelial cell growth supplement (ECGS) (Sigma-Aldrich). Cells are incubated in a 5% CO₂ environment at 37° C.

Cell media is changed every two days and washed with 1×PBS solution with every media change for the MC3T3-E1 cells. HUVEC cell media is aspirated and centrifuged at 1,500 rpm for 5 min in order to collect cellular debris that is essential for their proliferation. Cells are sub-divided by trypsin method. Cells are incubated with 0.25% trypsin/EDTA solution for 5-7 minutes until cells are no longer attached to the bottom of the culture vessel, stained with Trypan Blue exclusion dye and counted using a hemocytometer.

Seeding Efficiency.

Cellular Ti-6Al-4V alloy foam structures and metallographically polished flat Ti-6Al-4V alloy samples were seeded with pre-osteoblasts (200,000 cells/well in a 12 well cell culture plate) and incubated for 12 h at 37° C. in a CO₂ incubator to examine cell seeding efficiency. After incubating for 12 h, the specimens were removed from their respective wells with a trypsin solution and the cells remaining in each well were stained with Trypan Blue and counted using hemocytometer. The number of live cells estimated from the hemocytometer was subtracted from the total number of initially seeded cells, to obtain the number of cells that were seeded on each specimen. The seeding efficiency value was calculated as described in the following equation:

$$\% Seeding Efficiency = ( \frac{( Total number of cells seeded ) - ( Cells attached to the well )}{Total number of cells seeded} ) \times 100$$

MTS Viability Assay Protocol.

To study any lethal effect that DFM may have on MC3T3-E1 and HUVEC cells, an MTS viability assay (Promega) was performed. Hypoxia itself has not been reported to be cytotoxic to cells; however, some compounds may trigger a hypoxic response and produce a cytotoxic response, such as CoCl₂. In this experiment, the viability of both pre-osteoblasts and endothelial HUVEC cells are compared with exposures of 1:5 dilutions of DFM and CoCl₂ as a positive toxic response at various time exposures (24 h, 48 h, 72 h, 5 days & 14 days). In order to determine which concentrations of DFM are toxic to both MC3T3-E1 cells and HUVECs, an initial test is performed consisting of seeding 50,000 cells/well in a total volume of 200 μL of media with the 1:5 dilutions ranging from 2 mM to 640 nM of each compound. The starting stock solution of both hypoxia inducers is 10 mM. Cell media is changed every three days during the time of the experiment to avoid starvation related issues.

For cells grown in 3D printed foams, pre-osteoblast cells are seeded as previously described at a density of 200,000 cells/well in 12 well cell culture plates containing the 3D printed Ti-6Al-4V scaffold disks for the previously specified amount of time and dilutions of DFM. For this experiment, a negative control consists of cells growing of the tissue culture plate. After the desired periods of incubation, the scaffold is removed from the well with the cells attached to it to a new plate. Trypsinizing cells growing on foam samples has previously proven to produce inconsistent results and also requires extended trypsin incubation, leading to cellular detriment that may alter the viability results. It is for this reason that viability is measured without having to remove the cells from the foams by using the Vybrant® CFDA SE Cell Tracer Kit (Invitrogen). Fresh complete α-MEM media is added to the foams with cells. Subsequently each foam is treated with 1 μM of Vybrant® CFDA SE reagent A dissolved in reagent B, as indicated by the kit's specifications.

To perform the viability assay, 20 μL of the MTS stock solution (2 mg/mL) is added to each well. A negative control of 20 μL of the MTS stock solution added to 100 μL of medium is included. The 96 well plate is incubated at 37° C. for 4 hours and absorbance read at 490 nm. All experiments are performed in triplicates for statistical significance.

Combinatorial Dose of DFM & D(+) Glucose Viability Analysis in HUVECs and MC3T3-E1 Cells.

Cells that undergo hypoxia mimetic stress mediated by Hif-1α have been reported to increase their metabolic demands. To test how metabolic demands change in cells exposed to DFM, both HUVECs and MC3T3-E1 cells were exposed to 3.2 μM DFM, 5 mM D(+) glucose and a combination of 3.2 μM DFM+5 mM D(+) glucose for 24 hours. A negative control consisted of both cell types without treatment. The viability of cells was determined by MTS viability assay. The purpose of this approach is to determine whether D(+) glucose can be used as an additive in the hydrogel matrix to improve wound healing and help drive vascularization and osteogenesis process.

Cell Proliferation Analysis.

Cell proliferation is analyzed by counting cells by the trypan blue exclusion dye test. MC3T3-E1 cells are seeded at 50,000 cells/well and exposed to 1:5 dilutions of DFM and CoCl₂ in 200 μL total volume for 24 h, 48 h, 72 h, 5 days and 14 days. Cells were then trypsinized, stained, and counted. The population of live cells is determined by cell count. Each exposure is performed in statistical triplicates.

Fluorescent Microscopy.

Pre-osteoblasts seeded on foam structures were cultured for 7 days to test for similar cell behavior as reported by Nune et al. Cells were seeded at 200,000 cells/well in a 12 well plate, 1 mL total media volume and then stained with Hoechst 33342 cell nuclei dye. A second experiment was performed once the appropriate concentration of DFM was selected. Pre-osteoblasts are seeded at the previously described concentration in the same conditions but exposed to 3.2 μM DFM. Proliferation is observed after 21 days exposure. A second stain is performed with Vybrant® CFDA SE Cell Tracer Kit to visually determine the viability of cells without disturbing the layer of cells grown in the foams.

Because the scaffold is solid metal, light cannot be transmitted through it if fully dense. However, due to the foam nature of the scaffold, light may be transmitted though the medium and a visible image can be obtained if the metal section is thin enough. Despite the fact that a thin section makes fluorescent microscopy feasible, since the scaffold is 3D, focus is lost in the z axis and there is a lack of clarity for morphological details. Improvements on microscopy can be done by using Confocal Microscopy instead of Fluorescent Microscopy in order to view in more detail cellular morphology and microcapillary formation. Cell nuclei are stained with Hoechst 33342 dye at a working dilution of 1 μg/mL in complete media and incubated for 1 h. After the incubation period of time, the Ti-6Al-4V scaffold was flipped upside down to better observe the cells that grow on the uppermost section of the scaffold. Microscopy is performed in a Zeiss Axiovert 200 fluorescent microscope.

Induction of Angiogenesis in Foams Containing Hypoxia Mimetic ECM.

It has been previously reported that MC3T3-E1 pre-osteoblast cells undergo differentiation and proliferation when grown in porous foams in glutamine containing media [20,55]. It has been extensively reported that endothelial cells are able to form capillaries in gels when exposed to DFM [28]. In this experiment, a live angiogenesis monitoring experiment is performed in order to analyze capillarity maturation and cell survival. This analysis is achieved by staining HUVECs with PHK26 Red Fluorescent Dye (Sigma) and MC3T3-E1 cells with PKH67 Green fluorescent dye (Sigma). Both dyes stain cell membranes in an unspecific way, while maintaining cellular viability for an extended period of time. MC3T3-E1 cells are grown in foams as described above, but under hypoxic conditions (3.2 μM DFM). The Ti-6Al-4V foams are pre-incubated with pre-osteoblasts for a total of 7 days, with a negative hypoxia control being cells without any DFM. The cells grown on the foams and gel are monitored throughout the duration of this experiment. The foams are removed from the wells and placed in a 12 well plate and Matrigel® (Corning Life Sciences) will be added until the foam is completely covered. In order to observe microcapillarity formation upon exposure of HUVECs to DFM, the manufacturer specifications are followed. Briefly, Matrigel® is thawed according to specifications (4° C. overnight) and added to a 24 well cell culture plate and allowed to gelate also according to specifications (30-60 minutes at 37° C.). HUVECs (70-80% confluency) are tripsinized, re-suspended (1.2×10⁵ cells in 300 μL of complete media (10% FBS)) and treated with 10 μM of DFM. The cell suspension is incubated at 37° C., 5% CO₂ for 16 to 18 hours on the Matrigel coated plate for 16-18 hours. The formation of microcapillaries is measured by confocal microscopy.

Total Protein Content.

Cells are grown for 6 h, 24 h and 48 h in the presence of DFM at the previously specified 1:5 dilutions. After the desired time periods they are washed with cold 1×PBS and lysed with Radioimmunoprecipitation (RIPA) protein lysis buffer (0.2% Triton X-100+protease inhibitor cocktail) for 30 min in an ice bath. The total protein content in the cells is estimated using the Bradford spectrophotometric protein assay. Briefly, 10 μL of the cell lysate will be mixed with 100 μL of Bradford Reagent (Sigma) and re-suspended to homogenize the solution on a 96 well plate. The absorbance will be measured at 595 nm. A standard plot of absorbance as a function of bovine serum albumin (BSA) concentration will be obtained to determine the concentration value of the experimental and control samples.

Western Blot Analysis.

To further quantify the expression of specific proteins, a western blot analysis is performed for Hif-1α and VEGF₁₆₅. The western blot analysis was made using the cell lysate previously described. A Sodium Dodecyl Sulfate-Polyacrylamide Gel Electrophoresis (SDS-PAGE) was made (7.5% acrylamide for Hif-1α (93 kDa) and 12% acrylamide for VEGF₁₆₅ (38 kDa)). The separated proteins are then transferred to a PVDF membrane. The Hif-1α antibody at a 1:1,000 dilution in 1% BSA in 1×TBST buffer was incubated for one hour at room temperature, followed by three 10 min washes with TBST. A secondary anti-rabbit IgG HRP conjugated antibody was incubated for 30 minutes at room temperature, followed also by subsequent washes with TB ST. The immuno-blot is then revealed by the ECL method in film.

Cell Morphology and Adhesion.

Scanning Electron Microscopy is used to study the morphology, pore interconnectivity, and in-growth of pre-osteoblasts upon exposure to DFM. Cells are grown for 7 and 14 days. The negative control for this experiment is cells grown on foam samples without any hypoxic inducing molecules. The cell-seeded samples are fixed with 2.5% glutaraldehyde in 0.1 M cacodylate buffer pH 7.4 for 20 min, rinsed with PBS, dehydrated with a graded series of ethanol (25-100%) and critical point dried. Prior to examination of samples via SEM (Hitachi S-4800), the samples were sputter-coated with 10 nm of palladium to ensure sample conductivity and improve image resolution. The calcium-content in the ECM is estimated after the DFM exposure via Energy Dispersive X-Ray Analysis (EDAX) in the SEM.

Osteoblast Differentiation by Alkaline Phosphatase Activity (ALP).

Because osteoblasts express Alkaline Phosphatase (ALP) significantly, its activity can be used as a measure for cellular differentiation of pre-osteoblasts. The cell lysate obtained after treating with RIPA lysis buffer is used to determine the alkaline phosphatase activity, using ALP assay kit (Quantichrom). It has been previously reported that cell differentiation into calcium mineralizing MC3T3-E1 cells is inhibited under lack of L-glutamine in the growth media. A control for cellular differentiation is a lysate of cells grown in L-glutamine rich media (50 mM). The absorbance at 405 nm is measured via a spectrophotometer in a 96-well microplate reader. The ALP activity (expressed as micromoles of converted p-nitrophenol/min) was normalized by total intracellular protein synthesis (determined as described in the total intracellular protein content protocol and expressed as micromoles of p-nitrophenol per minute per milligram protein). ALP activity of osteoblasts cultured without any DFM and Tissue Culture Plate (TCP) served as controls.

Alizarin Red S (ARS) Mineralization Assay.

Because it is expected that pre-osteoblasts differentiate into osteoblasts under a hypoxic condition, cells should mineralize calcium in the ECM. The study of mineralization of ECM can be viewed as a measure or potential for bone formation. In this regard, Alizarin Red Assay (ARS) (Sigma-Aldrich) is used qualitatively and quantitatively to indicate the presence of calcium in the matrix. Cells are grown on foams at the selected optimum DFM concentration for 48 hours. The negative control for this experiment is cells grown in 3D printed foams in complete media with no added glutamate. After incubation, the samples are washed carefully with PBS without disturbing the cell layer and fixed with 4% paraformaldehyde for 30 minutes. The 3D foam structures were then stained with alizarin red (pH˜4.1) in the dark and at room temperature for 30 minutes. Samples are qualitatively studied via light microscope after rinsing with PBS. The calcium nodules should appear red. To conduct quantitative analysis, 10% cetyl pyridinium chloride will be added to remove the alizarin red stain. The absorbance of the solution is measured at 570 nm OD with a spectrophotometric microplate reader.

Statistical Analysis of Data.

The data is normalized with respect to control experiments and expressed as the mean of at least three replicates standard deviation (SD). Three sets of experiments are carried out for each experimental run. Statistical analysis is performed using a one way analysis of variance with 95% confidence interval.

An immediate issue that needs to be addressed is to determine the correct concentration of DFM needed to promote osteogenesis, bone formation and osteoblast differentiation coupled with angiogenesis and endothelial cell quiescence. A concentration of DFM needs to be chosen to promote these effects in both cell lines.

2. Results

Material Fabrication, Preparation and Characterization.

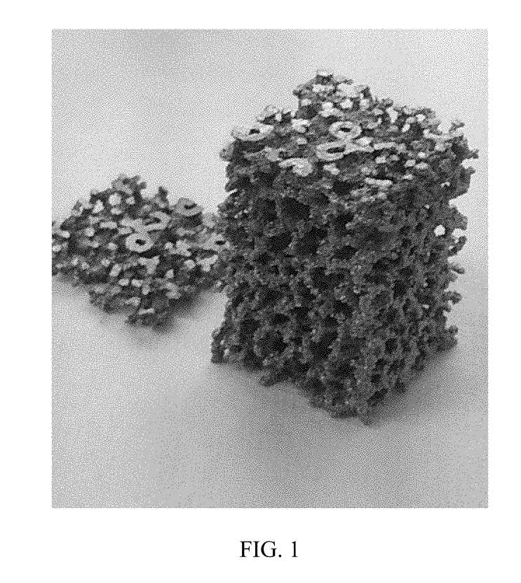

Ti-6Al-4V foams were successfully synthesized and characterized (FIG. 1). The structure in question has a density of 1.77 g/cm³ with 60% porosity and a modulus of 18 GPa. The average pore size was 350 μm in diameter, estimated by SEM analysis. Larger pores (outer portion of the foam) averaged 500 μm in diameter. The smaller pores (inner portion of the foam) averaged 200 μm in diameter.

Seeding Efficiency.

The efficiency of cell seeding in porous surfaces is directly related to the porosity of the material. A higher degree of porosity leads to a more limited surface area in which cells may grow and proliferate. Unlike mesh structures, foams are randomized and do not follow a structural pattern. Therefore, the seeding efficiency of a foam surface is related to the thickness of the sample. A thicker sample will yield a higher seeding efficiency value. A 0.5 cm thick 60% porous Ti-6Al-4V foam disk yields ˜58% seeding efficiency value.

MTS Viability Assay.

The evaluation of the viability of cells grown under hypoxia mimetic conditions must be analyzed in order to determine the appropriate amount of stress that the cells are undergoing, without promoting a cytotoxic effect. The degree of cellular metabolism needs to be assessed, whether cells are grown on a plastic tissue culture plate, a porous metal surface, or a 3D ECM matrix. This experiment serves the purpose of examining the optimal environmental conditions in which cells are stimulated to activate survival signals. The compounds tested include a known toxic hypoxia mimetic (CoCl₂) and the proposed PHD-2 inhibitor at 1:5 dilutions ranging from 2 mM to 640 nM of each compound.

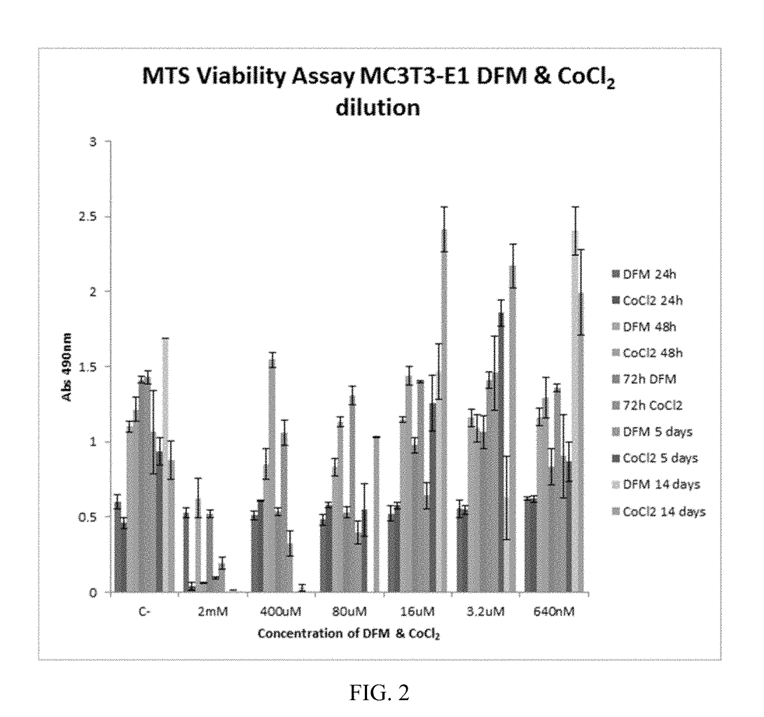

High doses of both compounds decrease cell viability drastically. In most cases, particularly at high concentrations CoCl₂ completely eradicated all cells present in the wells (FIG. 2). The LD₅₀ of CoCl₂ is not known for pre-osteoblast or osteoblast cell populations. Notably, MTS metabolism greatly increases for cells exposed to a hypoxia mimetic condition in periods of time equal or longer than 72 hours for both DFM and CoCl₂. This is in accordance to previous reports (Victor, et al., Cardiovascular Research 2008, 78:203-12) which show that near 10 μM concentrations of DFM can stimulate a survival response. Increased viability at 72 hours of exposure can be attributed to the pre-osteoblast's population doubling time. It is important to mention that, though the goal of this research is to create a hypoxia mimetic environment in the implant, the true levels of oxygen in the environment are not being depleted. Depletion of oxygen de-regulates mitochondrial production of cellular energy. The increased expression of Hif-1α and VEFG does not necessarily result in the de-regulation of mitochondrial energy production processes. Previous reports demonstrate that Glutathione S Transferase (GST) levels do not decrease with increased Hif-1α expression and the uptake of glutamine and glucose significantly increases (Zhanga et al., Toxicology and Applied Pharmacology 2016, 301:50-60). Excess glucose is required so that cells may endure the hypoxic reprograming.

D(+) Glucose Augments Viability Faster in HUVECs but not in MC3T3-E1.

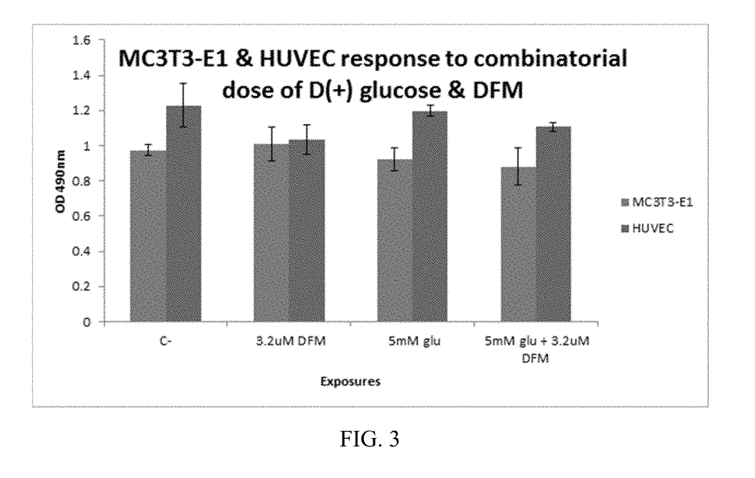

It has been previously suggested that the storage of glycogen increases in transplanted cells in bone tissue (Zhanga et al., Toxicology and Applied Pharmacology 2016, 301:50-60). The metabolic demand of glucose increases in cells with an activated Hif-1α program. To understand the effects that D(+) glucose may have on individual cell type populations, both HUVECs and MC3T3-E1 cells were exposed to treatments of 3.2 μM DFM, 5 mM glucose, and a combination of the both for 24 hours. The viability of cells was measured by MTS (FIG. 3).

No significant decrease in viability in either cell line after exposures were observed. However, HUVECs seem to have a more positive response to the additional glucose than the MC3T3-E1 cells. This may be explained due to HUVECs being more sensitive to oxygen sensing reprograming than MC3T3-E1 cells, increasing metabolic demands more significantly. This approach suggests that D(+) glucose may be used as a potential additive in the gel matrix to help cells enhance a wound healing response and to better tolerate stress.

Proliferation Analysis.

A cell count analysis is required in order to determine cellular proliferation. Cellular metabolism of MTS is not necessarily a direct measure of an increase in proliferation. Therefore, cell count experiments were performed by trypan blue exclusion dye method for MC3T3-E1 cells. A 24 hour exposure to both DFM and CoCl₂ dilutions reveals a toxic response to high concentrations of 2 mM. Despite seeing no cellular growth in any of the 2 mM CoCl₂ wells, there seems to be no immediate loss of cellular proliferation in the remaining wells. The wells containing cells exposed to DFM however, display constant cell population numbers, with a slight decrease in numbers when compared to control cells that were not exposed to any drug at 24 hours. The MTS analysis for cells exposed to both DFM and CoCl₂ for 24 hours shows almost no variation in cellular metabolism for the different amount of drug concentrations. However, given that the cells exposed to CoCl₂ are in fewer numbers and the MTS metabolism remains constant, it could be interpreted that these cells are undergoing stress and may be expressing cellular survival signaling.

Given that there is no immediate change in cellular population numbers, but metabolism of MTS readily increases, it can be assumed that cells exposed to both DFM and CoCl₂ are expressing survival signaling pathways. This can be analyzed by western blot analysis of p-Akt. Interestingly, more extended time exposures for DFM at high concentrations reveals an obvious decrease in cellular proliferation. Despite this noticeable decrease, proliferation does not reach untraceable levels, compared to CoCl₂ exposed cell populations. An observable increase in proliferation can be especially seen in cells exposed to the lowest concentrations of both DFM and CoCl₂ (3.2 μM and 640 nM). These results prove to be of significant importance given that it has been previously reported that HUVECs undergo angiogenesis at approximately 10 μM DFM (Victor, et al., Cardiovascular Research 2008, 78:203-12). The data obtained from suggests that both these cell types can be co-cultured, exposed to low concentrations of DFM (10-1 μM) and achieve a hypoxia mimetic response that can trigger survival signaling pathways.

Fluorescent Microscopy.

As previously shown by Murr et al., MC3T3-E1 cells grow and proliferate in Ti-6Al-4V foams of varying porosity, and a Hoechst 33342 staining in this case yielded similar results. Once the appropriate concentration of DFM was determined, MC3T3-E1 cells were seeded and grown in the Ti-6Al-4V foam for 21 days exposed to 3.2 μM DFM in a 12 well plate. Hoechst 33342 cell nuclei staining shows that pre-osteoblasts are proliferating in the foam under a hypoxia mimetic environment. A second staining was performed with the Vybrant® CFDA SE Cell Tracer Kit to visually determine the degree of cellular viability in the foam.

When HUVECs experience hypoxic stress, VEGF is secreted, and in turn, this signals neighboring cells to undergo a migration process to form microvasculature. In this experiment, the levels of secreted VEGF were analyzed by fluorescent microscopy techniques by staining VEGF with a fluorescently marked antibody.

Induction of Angiogenesis in Foams Containing Hypoxia Mimetic ECM.

The capacity an implant has to enable the formation of vascular structures is important, and therefore a requisite in order to consider the structure as a living implant. In this experiment, angiogenesis is induced in endothelial cells through exposure to DFM while grown in a 3D collagen based hydrogel. The hydrogel was polymerized in the presence of the printed Ti-6Al-4V foam. Seeing as it has been previously shown that HUVECs exposed to DFM undergo a reorganization process to form capillarity in Matrigel® (Victor, et al., Cardiovascular Research 2008, 78:203-12; Nune et al., J Biomed Mater Res Part A 2014, 00A:000-000), the purpose of this experiment is to analyze whether this process could be interrupted by the presence of metal foams. This experiment was designed to study the formation of capillarity by fluorescent microscopy for an extended period of time while maintaining cell viability. To achieve this, HUVEC membrane was stained with PKH26 (red fluorescence) whereas MC3T3-E1 cell membrane was stained with PKH67 (green fluorescence). Once successful staining was confirmed, Ti-6Al-4V foams were seeded with 2×10⁵ fluorescently labeled MC3T3-E1 cells and incubated for 12 hours to allow cellular adherence. After expiration of this time, Matrigel® was added to the foam containing the pre-incubated MC3T3-E1 cells. After an 18 hour period, HUVECs that were exposed to 2 mM DFM readily formed capillaries, suggesting the success of the approach. In the control setting (no DFM), these capillaries were not apparent. The integrity of the capillaries could not be measured. However, the capillaries seem to maintain their structure for an extended period of time. This can be measured because the cells used in this assay fluoresce without having to fix cells or stain with toxic dyes. The branching of these structures seems apparent, and it is consistent with previous reports (Victor, et al., Cardiovascular Research 2008, 78:203-12). After the confirmation of the formation of vascular networks in Matrigel, MC3T3-E1 cells staining and survival were analyzed.

After successful confirmation of the presence of MC3T3-E1 cells on foams and gels, HUVECs were then added to the well containing foam+gel with pre incubated MC3T3-E1 cells. HUVECs were added according to the Matrigel's manufacturer specifications. As previously mentioned, the experimental setting were HUVECs treated with 2 mM DFM at the moment of cellular resuspension.

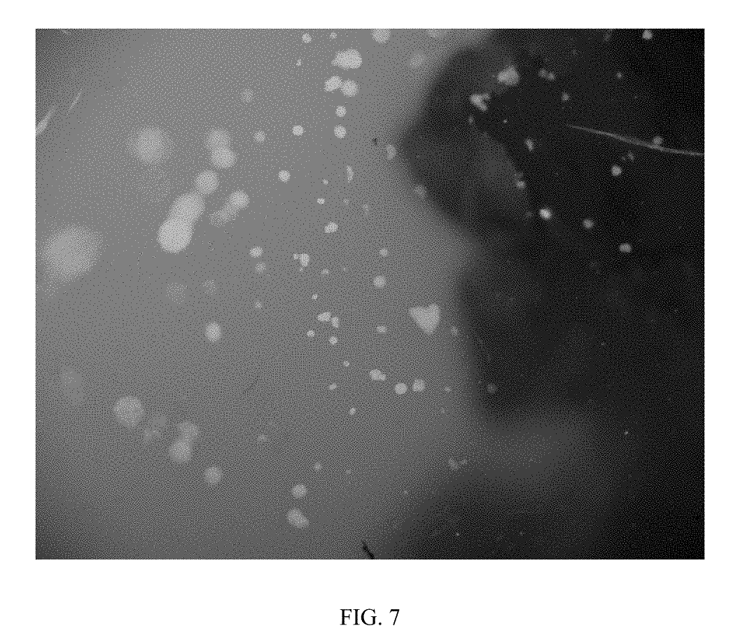

Fluorescent microscopy becomes challenging when analyzing 3D cell cultures, while solid foams also add complexity to the analysis. Vascular structures can form in foams with an ECM-like hydrogel matrix (FIG. 7). This result is central to the demonstration that vascularization is achievable in these materials, and supports the hypothesis that a foam does not present a physical barrier that hinders this biological process.

Total Protein Content.

In order to assess the total levels of protein being synthesized by cells exposed to DFM, a Total Protein Quantification analysis was performed in both HUVECs and MC3T3-E1 cells to show that protein levels do not decrease with DFM exposure. No obvious changes are observed in the levels of total protein synthesized by the endothelial cells. However, this analysis will further provide information as to the rate of cellular differentiation in pre-osteoblasts exposed to DFM.

Hif-1α and VEGF₁₆₅ Western Blot Analysis of HUVECs Exposed to DFM.

It has been previously reported that Hif-1α is barely detectable in HUVECs under normoxia. However, under the presence of low oxygen concentrations (pO₂<5%), intracellular levels of Hif-1α begin to increase (Victor, et al., Cardiovascular Research 2008, 78:203-12). This effect has been shown in HUVECs under DFM treatment. Here we can observe a relation between the administered concentration of DFM and the levels of intracellular Hif-1α. The cells barely express Hif-1α under normoxia, whereas high doses (2 mM) of DFM notably increase Hif-1α expression. Regardless of the amount of time of exposure to DFM, the production of Hif-1α maintains its stability. Cells exposed to DFM for 48 hours are still producing definitive bands of total intracellular Hif-1α. These results demonstrate the ability of DFM to maintain the hypoxia mimetic response.

Cell Morphology and Adhesion.

The distribution of cellular growth on the implant was analyzed by SEM imaging. SEM provides the advantage of analyzing the porous surface in which the pre-osteoblasts grow. It has been previously shown that MC3T3-E1 cells undergo cell differentiation when grown in foams of various densities under pre-osteoblast differentiation media (α-MEM 10% FBS+Glutamine) (Murr et al., J Mech Behav Biomed Mater. 2011, 4(7): 1396-411).

An SEM analysis was made in the same foam, but under a hypoxic mimetic environment with DFM. As previously shown, MC3T3-E1 cells undergo a drastic morphological change under the presence of DFM. The cells display an “elongation” which imparts on them a fibroblastic morphology.

Alkaline Phosphatase Differentiation Assay.

Because of the drastic morphological change on the pre-osteoblast cells, while maintaining viability, it is suspected that the hypoxia mimetic stress indices cellular differentiation.

Alizarin Red S Mineralization Assay.

It has been previously reported that MC3T3-E1 cells undergo cellular differentiation into osteoblasts in foams and other Ti-6Al-4V surfaces, and that they are able to mineralize calcium when grown in these surfaces (Murr et al., J Mech Behav Biomed Mater. 2011, 4(7):1396-411). Under ARS staining, the calcium nodes synthesized by the cells are apparent, particularly for lower DFM concentrations. The cells underwent evident morphological changes, as previously seen under DFM exposures.

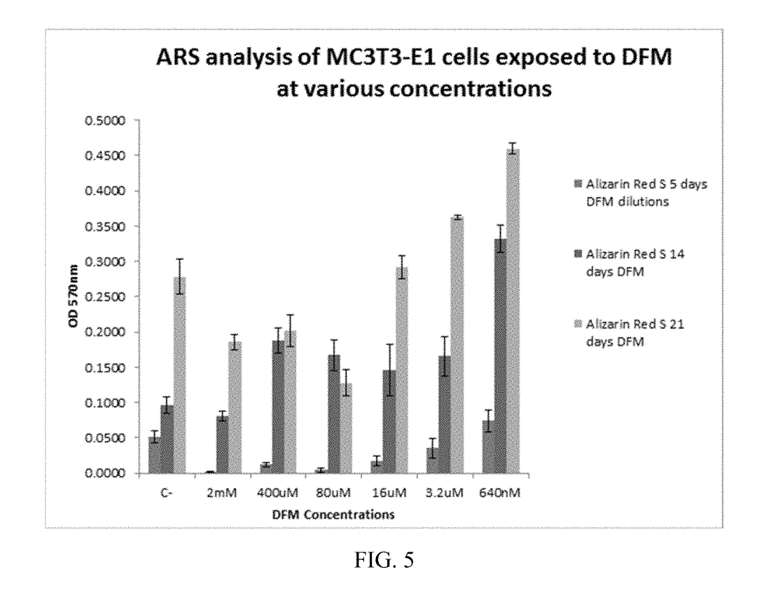

The synthesis of hydroxyapatite can be interpreted as a sign of pre-osteoblast differentiation given that the cell's phenotype expresses forms of mineralized calcium nodes. Despite seeing calcium nodes as early as 7 days, the production of hydroxyapatite is small in quantity, which correlates with previous reports (Murr et al., J Mech Behav Biomed Mater. 2011, 4(7):1396-411). However, synthesis of this main component of bone structure is mostly observed at lower concentrations of DFM. This data compliments that of cellular viability and proliferation in which we see increased metabolism, while maintaining cellular population at lower DFM concentrations, suggesting that said concentrations of DFM are sufficient to stimulate a desired effect of bone formation. Not only are the cells proliferating, but differentiating into a phenotype that promotes structural bone component formation. At 14 days of exposure to DFM the calcium nodes begin to look more obvious in all the wells when compared to a 7 day exposure. In all of the wells the levels of mineralized calcium seem apparent but they are most notable in the lower DFM concentration exposures. The highest expression of mineralized calcium can be observed at the 640 nM DFM exposed cells for 21 days (FIG. 5). Notably, in every instance, the expression of hydroxyapatite is always in greater amount for the cells exposed to 640 nM DFM when compared to control cells. This leads to the conclusion that DFM in fact increases the production of mineralized calcium.

It has been previously shown that MC3T3-E1 cells undergo cellular differentiation and successfully synthesize hydroxyapatite when grown on 3D printed Ti-6Al-4V foams (Murr et al., J Mech Behav Biomed Mater. 2011, 4(7):1396-411). Here, it is demonstrated that cellular production of hydroxyapatite can be enhanced by addition of 3.2 μM DFM.

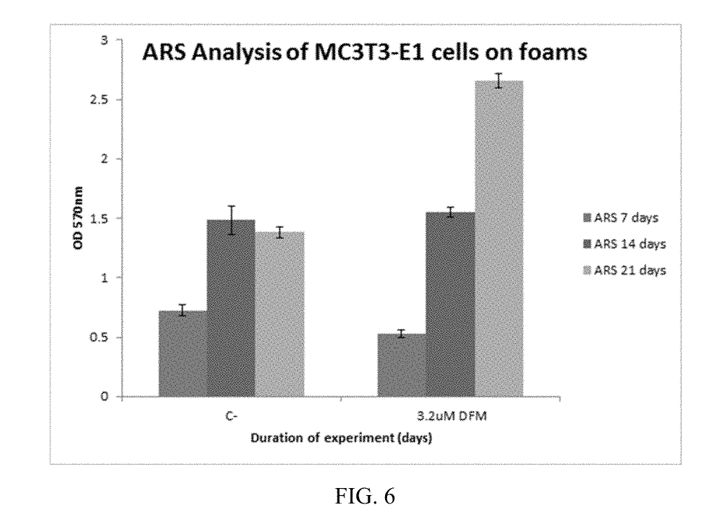

Initially, there is a definitive amount of hydroxyapatite being synthesized by the osteoblasts in both cases. Cells exposed to DFM express comparatively similar amounts of hydroxyapatite to control cells. Regardless of the hypoxic mimetic stress, where cells may be metabolically challenged to survive, the synthesis of hydroxyapatite does not seem to be suppressed. This can also be observed at longer time exposures of 14 days, the synthesis levels of hydroxyapatite seem to increase on par in both settings. After 21 days of exposure, the amount of cellular hydroxyapatite dramatically increases on the foam with cells exposed to DFM (FIG. 6). The foam containing cells exposed to 3.2 μM DFM displays a regular coating of hydroxyapatite (orange), and in considerably larger amounts than the control grown without DFM.

### SUMMARY OF RESULTS

| Experiment | Results |
|---|---|
| Seeding Efficiency | A seeding efficiency of 58% was achieved on a |
| | 0.5 cm thick 60% porous foam disk |
| MTS Viability | Cellular viability does not decrease in MC3T3- |
| | E1 cells exposed to DFM. Viability increases |
| | with lower DFM concentrations, as well as |
| | CoCl₂. |
| Cell Proliferation | Proliferation of MC3T3-E1 cells is affected at |
| Analysis | high DFM concentrations, but is increased in |
| | lower concentrations. |
| Fluorescent | MC3T3-E1 cells grow and proliferate on the |
| Microscopy | foam alloy when exposed to low DFM |
| Analysis | concentrations for a long period of time. |
| Induction of | HUVECs readily undergo migration to form |
| Angiogenesis | capillarities in gels and in foams with gel |
| in Foams | mixtures when exposed to DFM. |
| Total Protein Content | Total protein contents were analyzed and |
| | further used in the Western Blot analysis and |
| | the ALP analysis. |
| Western Blot Analysis | Hif-1α levels increased with increasing DFM |
| | concentration in HUVECs. |
| Cell Morphology & | Visual confirmation of cells growing in foams |
| Adhesion | through SEM analysis has been confirmed. |
| Osteoblast | |
| Differentiation | |
| by ALP activity | |
| Alizarin Red S | DFM enhances the cellular production of |
| Mineralization | hydroxyapatite both in cells grown in plates |
| Assay | and in foams. |

1. A bone replacement implant comprising:
(a) a three dimensional support; and
(b) a hydrogel matrix comprising a hypoxia inducer and glucose;
wherein the implant is capable of promoting vascularization and osteogenesis.

2. The implant of claim 1, wherein the three dimensional support is a scaffold of Ti-6Al-4V.

3. The implant of claim 2, wherein the scaffold has a porosity of 50 to 70%.

4. The implant of claim 2, wherein the scaffold has an average pore size of 200 to 500 μm.

5. The implant of claim 2, wherein the scaffold has a thickness of 0.25 to 5 cm.

6. The implant of claim 2, wherein the scaffold has a density of 1 to 2 g/cm².

7. The implant of claim 1, wherein the hydrogel comprises proteins of the extracellular matrix.

8. The implant of claim 1, wherein the hydrogel comprises natural, synthetic, or natural and synthetic polymers.

9. The implant of claim 8, wherein the natural polymers are one or more of polyhyaluronic acid, alginate, polypeptides, collagen, elastin, polylactic acid, polyglycolic acid, or chitin.

10. The implant of claim 8, wherein the synthetic polymers are one or more of polyethylene oxide, polyethylene glycol, polyvinyl alcohol, polyacrylic acid, polyacrylamide, poly(N-vinyl-2-pyrrolidone), polyurethane, or polyacrylonitrile.

11. The implant of claim 1, wherein the hydrogel further comprises one or more growth factors.

12. The implant of claim 1, wherein the hydrogel further comprises an antibiotic.

13. The implant of claim 1, wherein the hypoxia inducer is deferoxamine mesylate (DFM).

14. The implant of claim 13, wherein the DFM is present at a concentration of about 2 to 5 μM.
